(19) United States
(12) Patent Application Publication
(10) Pub. No.: US 2025/0073724 A1
(43) Pub. Date: Mar. 6, 2025

(54) DETECTION DEVICE AND LIGHT GUIDE DETECTION METHOD THEREOF

(71) Applicant: HANGZHOU ZHILINGLONG BIOTECHNOLOGY CO LTD, Hangzhou City (CN)

(72) Inventors: Xing SU, Hangzhou City (CN); Fulin XING, Hangzhou City (CN)

(21) Appl. No.: 18/819,183

(22) Filed: Aug. 29, 2024

(30) Foreign Application Priority Data
Aug. 29, 2023 (CN) 202311099895.4

(51) Int. Cl. B01L 7/00; G01N 21/64

(52) U.S. Cl. CPC B01L 7/52; G01N 21/6428; B01L 2200/0647; B01L 2300/041; B01L 2300/0654; B01L 2300/1805; G01N 2021/6432; G01N 2021/6439

(57) ABSTRACT

The invention relates to biochemical reactions, in particular to a detection device and a light guide detection method thereof. The detection device comprises: a reaction vessel providing a reaction space and having a first end and a second end opposite the first end, wherein the first end of the reaction vessel is an open end and the second end of the reaction vessel is a closed end; a reaction chip arranged inside the reaction space and located at a first end of the reaction vessel; a cover detachably provided on the first end of the reaction vessel to close the reaction space; and a light guide assembly passing through the cover from the outside of the reaction space into the inside of the reaction space and connected to the reaction chip. Compared with direct-irradiation of the reaction chip by the excitation light, the invention adopts a light guide assembly-evanescent wave method which effectively avoids the influence of light source irradiation on the whole reaction mixture, reduces phototoxicity, effectively shields from background noises, provides higher signal-to-noise ratios and increases the reliability of detection results.

### RELATED APPLICATION

This application claims priority from CN Application No.: 202311099895.4 titled, “Detection device and light guide detection method thereof” filed on Aug. 29, 2023, which is incorporated herein by reference in its entirety.

### TECHNICAL FIELD

The invention relates to biochemical reactions, in particular to a detection device and a light guide detection method thereof.

### SEQUENCE LISTING

The instant application contains a Sequence Listing submitted electronically in XML format which was created on Aug. 7, 2024, and modified on Aug. 9, 2024, using WIPO ST.26 sequence listing. The said XML filed is named as HZBL-002-01US.xml and has a size of 57.9 KB. The XML format is hereby incorporated by reference in its entirety.

### BACKGROUND

Several amplification devices have been developed to amplify nucleic acid target molecules. In addition, detection devices have been developed that allow detection of target molecules. However, it is desirable to perform multiplex amplification and multiplex detection of multiple types of target molecules simultaneously in the same liquid phase reaction mixture.

There are techniques currently available that employ annular convection chambers for PCR amplification, microarrays for detection, and conventional optical methods for chip-reading. However, such a technology has complex reaction device structure, and is complicated in processing the consumable of the annular convection chamber that is also difficult to seal and thus prone to product contamination; the technology has low detection throughput and is hard to achieve high-throughput sample detection; the microarray of the technology has low fabrication efficiency, and is difficult to realize mass production; the technology has low efficiency of target molecule amplification, in which polymerases without an exonuclease activity are often used, rendering low fidelity and low efficiency in amplifying multiple relevant target molecules; furthermore, the excitation light source of such a technology needs to be introduced externally, and direct light excitation causes low signal-to-noise ratios, resulting in less reliable results.

Therefore, a detection device is needed, which not only can carry out multiplex amplification and multiplex detection of a plural types of target molecules in the same liquid phase reaction mixture, but also can reduce the possibility of product contamination, increase detection throughput, reduce usage costs, effectively shield from background noises, provide a higher signal-to-noise ratio, and increase the reliability of detection results.

### DISCLOSURE OF INVENTION

In order to solve problems existing in the prior art, the present invention provides a detection device and a light guide detection method thereof, which not only can carry out multiplex amplification and multiplex detection of a plural types of target molecules in the same liquid phase of a reaction mixture, but also can reduce the possibility of product contamination, increase the detection throughput, reduce usage costs, effectively prevent the entire reaction mixture from being affected by light source irradiation to reduce the phototoxicity, effectively shield from background noises, provide higher signal-to-noise ratios, and increase the reliability of detection results. A target molecule in the present invention refers to a target nucleic acid molecule or a nucleic acid fragment to be detected, which may be a nucleic acid molecule fragment formed by amplifying the target nucleic acid molecule to be detected, or may be a target nucleic acid molecule to be detected which has not been amplified.

The invention provides a detection device, comprising:
a reaction vessel providing a reaction space and having a first end and a second end opposite the first end, wherein said first end of said reaction vessel is an open end and said second end of the reaction vessel is a closed end, wherein the said reaction vessel is used for nucleic acid amplification reactions;
a reaction chip being arranged inside said reaction space and at said first end of said reaction vessel, wherein a plural types of nucleic acid probes complementary to a plural types of target molecules, respectively, are immobilized on a reaction surface of said reaction chip, wherein the nucleic acid probes are used to detect nucleic acid molecules formed by amplifying said target molecules in said reaction vessel;
a cover being detachably provided on said first end of said reaction vessel to close said reaction space;
a light guide assembly passing through said cover from the outside of the reaction space into the inside of said reaction space and being connected to said reaction chip.

In one embodiment of the invention, the light guide assembly comprises an optical fiber, a light converter and a coupler, wherein said optical fiber is connected to said light converter, and said light converter connects excitation light to said reaction chip via said coupler.

In one embodiment of the invention, the light guide assembly further comprises a condenser lens and a light source, wherein said condenser lens is capable of guiding excitation light emitted by said light source into said optical fiber.

In one embodiment of the invention, the light source is a laser light source, a LED light source, or other light sources.

In one embodiment of the invention, the reaction chip is fixed vertically or horizontally to the cover via said light guide assembly.

In one embodiment of the invention, the reaction chip, the cover and the light guide assembly are integrated together.

In one embodiment of the invention, the device further comprises a fluorescent signal detector, wherein said fluorescent signal detector detects a fluorescent signal on the reaction chip.

In one embodiment of the invention, the device further comprises a computing-control module, said computing-control module controls the light guide assembly and the fluorescent signal detector, and outputs the detection results of the detection device based on the detection of said fluorescent signal detector.

In one embodiment of the invention, said detection device further comprises:
a first heater located at said first end of said reaction vessel;
a second heater located at said second end of said reaction vessel.

In one embodiment of the present invention, said first heater and said second heater are a single heater, a dual heater, or a ring heater, respectively.

In one embodiment of the invention, said detection device further comprises one or more third heaters, said third heater located between said first end and said second end of said reaction vessel.

In one embodiment of the present invention, in the case where both said first heater and said second heater are heated, or in the case where said first heater and said second heater plus said third heater are heated, said reaction vessel can be heated so that the temperature at said first end of said reaction vessel is 30° C. to 75° C., and so that the temperature at said second end of said reaction vessel is 35° C. to 110° C.

In one embodiment of the present invention, said reaction vessel is a tubular structure, said first end and said second end of said reaction vessel are arranged opposite to each other in a length direction of said tubular structure, said first end and said second end of said reaction vessel are arranged concentrically or non-concentrically, and a cross sections of said first end and a cross section of said second end of said reaction vessel are the same or different.

In one embodiment of the invention, a cross section of said first end and a cross section of said second end of said reaction vessel consist of at least one of a curvilinear side and rectilinear side, respectively.

In one embodiment of the invention, the inner diameter or minimum side length of said cross section of said first end and said second end of said reaction vessel is 0.5 mm to 5 mm, the length of said tubular structure is 5 mm to 50 mm, and the volume of said reaction space is 5 μl to 5000 μl.

In one embodiment of the invention, said reaction surface of said reaction chip is directed in a radial direction of said tubular structure or in the length direction of said tubular structure and towards the second end of said reaction vessel.

In one embodiment of the invention, the number of the plural types of nucleic acid probes are 2, 3 to 10, 3 to 20, 3 to 30, 3 to 300, 3 to 3000, or 3 to 3000000.

In one embodiment of the invention, said plural types of nucleic acid probes are immobilized onto said reaction surface of said reaction chip in an in situ or ex situ synthesis manner.

In one embodiment of the invention, the first heater is integrated into the reaction chip.

In one embodiment of the invention, the plural types of target molecules comprise one or more of RNA molecules or DNA molecules, RNA fragments in an RNA genome or DNA fragments in a DNA genome, and variant structures in an RNA molecule or a DNA molecule.

In one embodiment of the invention, the plural types of target molecules comprise one or more of RNA virus nucleic acid molecules and DNA virus nucleic acid molecules, wherein the RNA virus comprises one or more of influenza A virus InfA, influenza A virus H1N1 2009, influenza A virus H3N2, human parainfluenza virus HPIV1, human parainfluenza virus HPIV2, human parainfluenza virus HPIV3, human parainfluenza virus HPIV4, human metapneumovirus hMPV, respiratory adenovirus AdV, respiratory syncytial virus RSV, bocavirus BoV, severe acute respiratory syndrome coronavirus SARS-COV, middle east respiratory syndrome coronavirus MERS-COV, and severe acute respiratory syndrome coronavirus 2 SARS-COV-2, wherein the DNA virus comprises one or more of human herpes viruses HSV-1, human herpes virus HSV-2, human herpes virus VZV, human CMV, human EBV, human HHV-6, HHV-7, and human herpes virus HHV-8.

In one embodiment of the invention, the plural types of target molecules are derived from humans, animals, plants, microorganisms or synthesized manually or chemically, wherein microorganisms include one or more of viruses, bacteria and fungi.

The invention further provides a light guide detection method for said detection device, the method comprising: injecting a reaction mixture, consisting of a reaction mixture with a test sample, into the interior of a reaction space provided by a reaction vessel, said test sample comprising one or more target molecules to be detected; emitting excitation light from a light source and guiding the excitation light to a reaction chip via a light guide assembly to generate an evanescent wave on a reaction surface of said reaction chip, such that nucleic acid molecules formed by amplifying one or more of the target molecules within the reaction vessel are capable of generating a fluorescent signal after hybridization with complementary nucleic acid probe molecules immobilized on the reaction surface of said reaction chip; detecting the fluorescent signals by a fluorescent signal detector; determining the types of one or more target molecules that hybridize with the complementary nucleic acid probe molecules based on the locations or types of said complementary nucleic acid probes where fluorescent signals are detected.

In one embodiment of the invention, said excitation light is directed into said reaction chip by a coupler at an angle of total internal reflection.

In one embodiment of the invention, said excitation light is coupled into an optical fiber.

In one embodiment of the invention, said evanescent wave propagates parallelly to said reaction surface of said reaction chip within a range from said reaction surface of said reaction chip to a first thickness, wherein the intensity of said evanescent wave decays exponentially with increasing thickness.

In one embodiment of the invention, said first thickness is 100 nm.

In one embodiment of the invention, said method further comprises heating said reaction vessel by a first heater and a second heater to enable a reaction mixture with a test sample to form convection flow between the first end and the second end of said reaction vessel under the action of thermal convection and to enable said one or more target molecules to be detected in said test sample to hybridize not only to the complementary primers in said reaction mixture for amplification, but also to the complementary nucleic acid probe molecules immobilized on the reaction surface of the reaction chip.

In one embodiment of the invention, said method further comprises heating said reaction vessel by one or more third heaters.

In one embodiment of the present invention, the heating operations of either the first heater and the second heater or both the first heater and the second heater plus the third heater are controlled separately such that the temperature at the first end of the reaction vessel is 30° C. to 75° C. and such that the temperature at the second end of the reaction vessel is 35° C. to 110° C.

In one embodiment of the invention, said reaction vessel is arranged vertically or obliquely.

In one embodiment of the present invention, when said reaction vessel is arranged obliquely, the angle between said reaction vessel and the vertical direction is 0° to 45° C.

In one embodiment of the invention, said reaction system comprises a primer and a DNA polymerase.

In one embodiment of the present invention, said DNA polymerase has a 3′→5′ exonuclease activity.

In one embodiment of the invention, a fluorescent signal is generated by direct excitation of fluorescent dyes, dye intercalation, fluorescence resonance energy transfer, or fluorescence dequenching.

In one embodiment of the invention, the type of nucleic acid probe is identified by a predetermined location of the nucleic acid probe on said reaction surface of said reaction chip.

As described above, the present invention has the following advantages:

According to present invention, the reaction chip for multiplex detection can be arranged inside a reaction vessel where thermal convection amplification reaction can take place, such that probe molecules hybridize with target molecules while a multiplex amplification is taking place and fluorescent signals from the reaction chip are being detected; there is only one liquid phase of the reaction mixture and one step of operation in the whole detection process. In addition, compared with the excitation light directly irradiating the reaction chip, the invention adopts the light guide assembly-evanescent wave method, which can effectively prevent the entire reaction mixture from being affected by the irradiation of the light source, and reduce phototoxicity, shield from background noises effectively, provide higher signal-to-noise ratio, and increase the reliability of detection results.

### DRAWINGS

FIG. 1A is a schematic diagram of the overall structure of a detection device according to one embodiment of the present invention.

FIG. 1B is a side view of a detection device according to one embodiment of the invention.

FIGS. 2A to 2B are schematic views showing the arrangement of a reaction chip in a detection device according to an embodiment of the present invention.

FIGS. 3A to 3C are schematic views showing, respectively, the integration of a reaction chip, a cover and a light guide assembly in a detection device according to an embodiment of the present invention.

FIG. 4A is a schematic view of a part of the structure of a detection device according to another embodiment of the present invention.

FIG. 4B is a schematic view of a part of the structure of a detection device according to still another embodiment of the present invention.

FIGS. 5A to 5B are schematic views showing, respectively, the integration of a first heater and a reaction chip in a detection device according to an embodiment of the present invention.

FIG. 6A to 6B are a principal diagram and schematic diagrams, respectively, of a light guide detection method for a detection device according to an embodiment of the present invention.

### DETAILED DESCRIPTION

Embodiments of the present invention are described below with reference to the accompanying drawings.

Reference will now be made in detail to exemplary embodiments, examples of which are illustrated in the accompanying drawings. When the following description refers to the accompanying drawings, the same numbers in different drawings refer to the same or similar elements, unless otherwise indicated. The implementations described in the following exemplary examples do not represent all implementations consistent with the invention. Rather, they are merely examples of device and methods consistent with aspects of the invention as detailed in the accompanying claims.

Terminologies used in the present invention are for the purpose of describing embodiments only and are not intended to be limiting of the invention. As used in this specification and the appended claims, the singular forms “a,” “an,” and “the” are intended to include the plural forms as well, unless the context clearly indicates otherwise. It should also be understood that the term “and/or” as used herein refers to and encompasses any or all possible combinations of one or more of the associated listed items. The word “comprising” or “comprises”, and the like, means that elements or items appearing before “comprising” or “comprises” are encompassed by the element or item recited after “comprising” or “comprises” and equivalents thereof, and that other elements or items are not excluded.

### Example 1

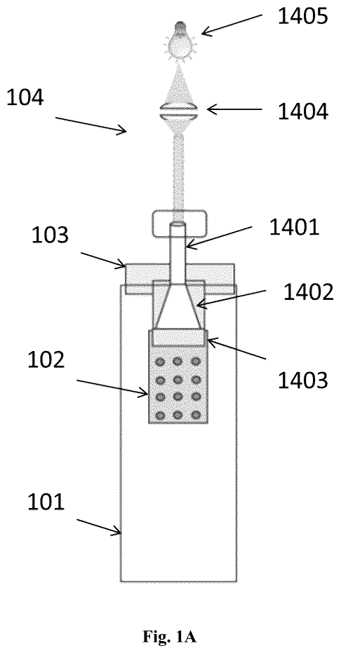

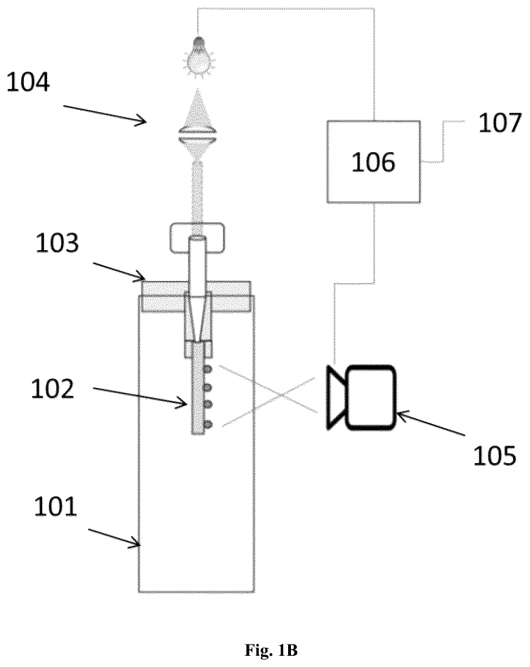

A first embodiment of the present invention provides a detection device. FIG. 1A is a schematic view of the overall structure of a detection device according to an embodiment of the present invention, and FIG. 1B is a side view of the detection device according to an embodiment of the present invention.

As shown in FIGS. 1A and 1B, the detection device may include a reaction vessel 101, a reaction chip 102, a cover 103, and a light guide assembly 104.

The reaction vessel 101 may also be referred to as a reaction test tube, the reaction vessel 101 may provide a reaction space, which may also be referred to as a reaction chamber, and the reaction vessel 101 may have a first end and a second end opposite the first end, wherein the first end of the reaction vessel 101 may be an open end and the second end of the reaction vessel 101 may be a closed end.

The cover 103 may be detachably provided on the first end of the reaction vessel 101 to close the reaction space, and the cover 103 may not be in contact with the reaction mixture in the reaction space, thereby greatly reducing the possibility of contamination due to product exudation.

In one embodiment, the reaction vessel 101 may be a tubular structure, such as a round tubular structure or a square tubular structure, which is more easily encapsulated than a reaction vessel of a sheet-like structure. The first end and the second end of the reaction vessel 101 may be arranged opposite to each other in the length direction of the tubular structure, and in FIGS. 1A and 1B, the first end of the reaction vessel 101 may also be referred to as an upper end of the tubular structure, and the second end of the reaction vessel 101 may also be referred to as a lower end of the tubular structure.

In one embodiment, the first end and second end of the reaction vessel 101 may be arranged concentrically or non-concentrically.

In one embodiment, the cross sections of the first end and second end of the reaction vessel 101 may be the same or different.

In one embodiment, the cross sections of the first end and second end of the reaction vessel 101 may be composed of at least one of a curvilinear side and rectilinear side, respectively.

In one embodiment, the cross sections of the first end and second end of the reaction vessel 101 may be a circle and a square, respectively. In other words, the cross sections of the first end and second end of the reaction vessel 101 may be circular, so that the reaction vessel 101 may have a circular tubular structure. Alternatively, the cross sections of the first end and second end of the reaction vessel 101 may be square, so that the reaction vessel 101 may be of square tubular structure. Alternatively, one of the first end and the second end of the reaction vessel 101 may be circular in cross section, and the other may be square in cross section, so that the reaction vessel 101 may be a profiled tubular structure.

In one embodiment, the inner diameter or minimum side length of the cross section of the first end and the second end of the reaction vessel 101 may be 0.5 mm to 5 mm, the length of the tubular structure may be 5 mm to 50 mm, and the volume of the reaction space may be 5 μl to 5000 μl.

In one embodiment, the reaction vessel 101 may be made of a heat resistant material, which may have a heat resistant temperature of, for example, greater than 150° C.

In one embodiment, the reaction vessel 101 may be made of a transparent material. As will be described in detail below, the transparent material may be suitable for use in a fluorescence signal detection.

Compared with the annular convection chamber, the tubular reaction vessel 101 is easier to process, for example, it can be formed in one step using a traditional injection molding process, has low production costs, and is more reliable to use.

The reaction chip 102 may be arranged inside the reaction space, and may be located at a first end of the reaction vessel 101. In other words, the reaction chip 102 may be located at the open end of the tubular structure, and in FIG. 1A and FIG. 1B, the reaction chip 102 may be located at the upper end of the tubular structure.

The size of the reaction chip 102 and its position at the first end of the reaction vessel 101 may be arranged so that a reaction mixture with a test sample injected inside can cover the reaction surface of the reaction chip 102 and can freely flow through the reaction surface of the reaction chip 102 by thermal convection to be described below.

In one embodiment, the reaction chip 102 may be made of a silicon material, a glass material, or a high molecular weight polymer material; it has low production costs, and is suitable for mass production.

In one embodiment, the reaction vessel 101 is an apparatus for nucleic acid amplification; plural types of nucleic acid probes complementary to the plural types of target molecules, respectively, may be immobilized on the reaction surface of the reaction chip 102, the nucleic acid probes are used to detect nucleic acid molecules formed by amplifying the target molecules in the detection device, and the nucleic acid probes may form a two-dimensional probe array so that labels of the nucleic acid probes of different types may be located at x-y coordinates. As an example, the nucleic acid probe at the (1, 1) position on the reaction surface may be preset as type A, and the nucleic acid probe at the (1, 2) position on the reaction surface may be preset as type B, and so on.

The molecules of the nucleic acid probes may be single-stranded, and when a certain probe molecule hybridizes with a complementary type of a target molecule in the test sample, double-stranded molecules may be formed, and then the single-stranded molecules and the double-stranded molecules may be distinguished by fluorescent signals. Thus, when it is determined that a known nucleic acid probe molecule at a certain position on the reaction surface hybridizes with a complementary target molecule to form a double-stranded structure molecule, the presence of the complementary target molecule in the test sample can be identified. In the case where a plurality of nucleic acid probes complementary to plural types of target molecules are immobilized on the reaction surface, it is possible to identify whether plural types of target molecules are present in the test sample, thereby achieving multiplex detection.

These nucleic acid probes may be immobilized on the reaction surface of the reaction chip 102 in an in situ synthesis, i.e., directly synthesized and immobilized, or ex situ synthesis, i.e., synthesized off site and followed by transferring and immobilization; there are also a number of known methods for synthesizing nucleic acid probes and surface immobilization.

In one embodiment, the number of the plurality of nucleic acid probes may be 2, 3 to 10, 3 to 20, 3 to 30, 3 to 300, 3 to 3000, or 3 to 3000000. As shown in FIG. 1A, the number of the plurality of nucleic acid probes may be 12, and may be formed as a 3×4 probe array.

In one embodiment, the reaction surfaces of the reaction chip 102 may be oriented in a radial direction of the tubular structure, i.e., the reaction chip 102 may be arranged vertically in the tubular structure. In another embodiment, the reaction surface of the reaction chip 102 may be oriented in the length direction of the tubular structure, i.e., the reaction chip 102 may be arranged horizontally in the tubular structure and toward the second end of the reaction vessel.

The light guide assembly 104 may pass through the cover 103 from the outside of the reaction space into the inside of the reaction space, and be connected to the reaction chip 102.

In one embodiment, the reaction chip 102, the cover 103 and the light guide assembly 104 may be integrated together, and the reaction chip 102 and the light guide assembly 104 may enter the tubular structure with the cover 103 when the reaction space is closed by the cover 103. As an example, the reaction chip 102 may be vertically fixed on the cover 103 via the light guide assembly 104, so that the reaction surface of the reaction chip 102 may face in the radial direction of the tubular structure when the reaction space is closed by the cover 103. As another example, the reaction chip 102 may be horizontally fixed on the cover 103 via the light guide assembly 104, so that when the reaction space is closed by the cover 103, the reaction surface of the reaction chip 102 may face the length direction of the tubular structure and toward the second end of the reaction vessel 101.

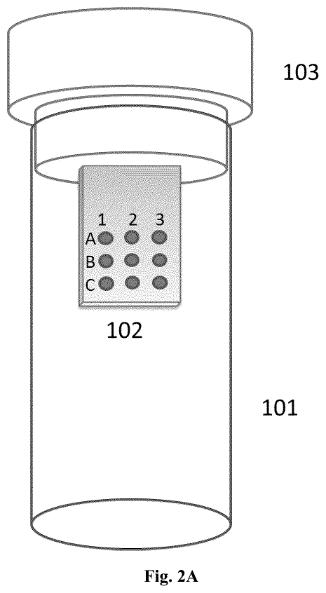

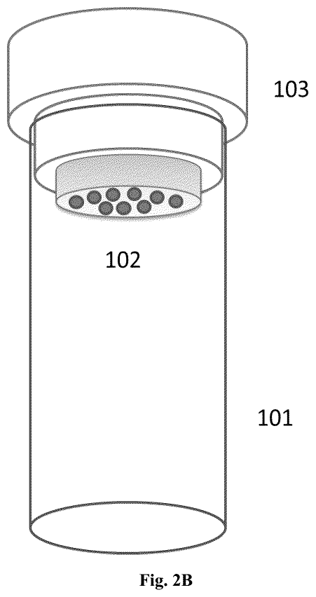

FIG. 2A to 2B are schematic diagrams illustrating an arrangement of a reaction chip in a detection device according to an embodiment of the present invention.

As shown in FIG. 2A, the reaction chip 102 may be vertically fixed to the cover 103 so as to be able to enter the tubular structure with the cover 103 when the reaction space is closed by the cover 103. The reaction surface of the reaction chip 102 may be oriented in a radial direction of the tubular structure.

As shown in FIG. 2B, the reaction chip 102 may be horizontally fixed to the cover 103 to be able to enter the tubular structure with the cover 103 when the reaction space is closed by the cover 103. The reaction surface of the reaction chip 102 may be oriented in the length direction of the tubular structure and toward the second end of the reaction vessel 101.

In one embodiment, the light guide assembly 104 may comprise an optical fiber 1401, a light converter 1402 and a coupler 1403, the optical fiber 1401 may be connected to the light converter 1402, the light converter 1402 may convert excitation light in the form of columnal light into sheet-light and be connected to the reaction chip 102 via the coupler 1403.

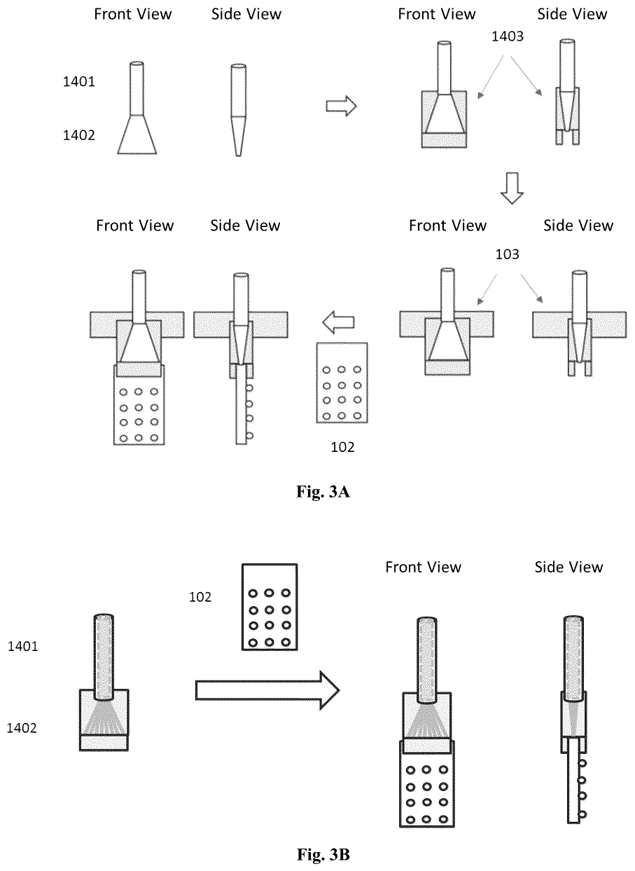

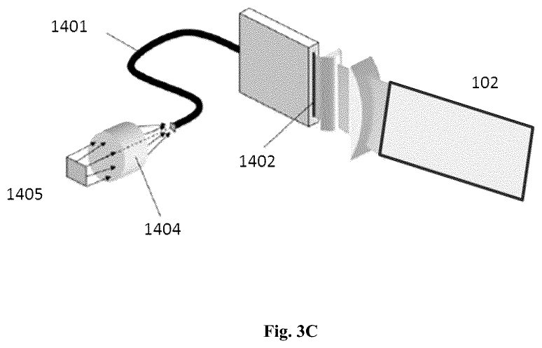

FIG. 3A to 3C are schematic views of integrated structures of a reaction chip, a cover and a light guide assembly in a detection device according to an embodiment of the present invention.

As shown in FIG. 3A, one end of the optical fiber 1401 may be first connected to one end of the optical converter 1402, then the coupler 1403 may be sleeved outside the optical converter 1402, then the other end of the optical fiber 1401 may be passed through the cover 103, and finally the reaction chip 102 may be sandwiched on the coupler 1403 and may be connected to the other end of the optical converter 1402, so that the reaction chip 102, the cover 103 and the light guide assembly 104 may be integrated together, and the optical fiber 1401 may be optically connected to the reaction chip 102 via the optical converter 1402.

As shown in FIG. 3B, the optical fiber 1401 may be a fiber bundle, and the optical converter 1402 may be a linear optical fiber group in which optical fibers may be arranged in a linear shape. The columnal light from the fiber bundle may be converted into an optical sheet by the linear fiber group, and the optical sheet may be irradiated to one side of the reaction chip 1402 and may be coupled to the light guide layer of the reaction chip 102 so that evanescent light is generated on the surface of the reaction chip 102.

As shown in FIG. 3C, the light converter 1402 may include a cylindrical lens or a spherical lens. The columnal light or spherical light may be converted into a sheet-light by a lens, which may be irradiated to one side of the reaction chip 1402, and may be coupled to a light guide layer of the reaction chip 102 so that evanescent light is generated at the surface of the reaction chip 102.

In another embodiment, the light guide assembly 104 may further include a condenser lens 1404 and a light source 1405, the condenser lens 1404 being capable of guiding excitation light emitted by the light source 1405 into the optical fiber 1401.

In one embodiment, the light source may be a laser light source, an LED light source, or other light sources.

The detection device may further include a fluorescent signal detector 105, and the fluorescent signal detector 105 may detect a fluorescent signal on the reaction chip 102. The principle of generating fluorescent signal will be detailed below.

In one embodiment, the fluorescence signal detector 105 may be a CCD camera, an EMCCD camera, a CMOS camera, or a light sensitive sensor (such as a photomultiplier tube), etc., and the collection and detection of the fluorescence signals may be optical scanning or imaging, etc.

The detection device may further include a computing-control module 106, and the computing-control module 106 may control various components in the detection device, such as the light guide assembly 104 and the fluorescence signal detector 105, and output a detection result 107 of the detection device based on the detection of the fluorescence signal detector 105.

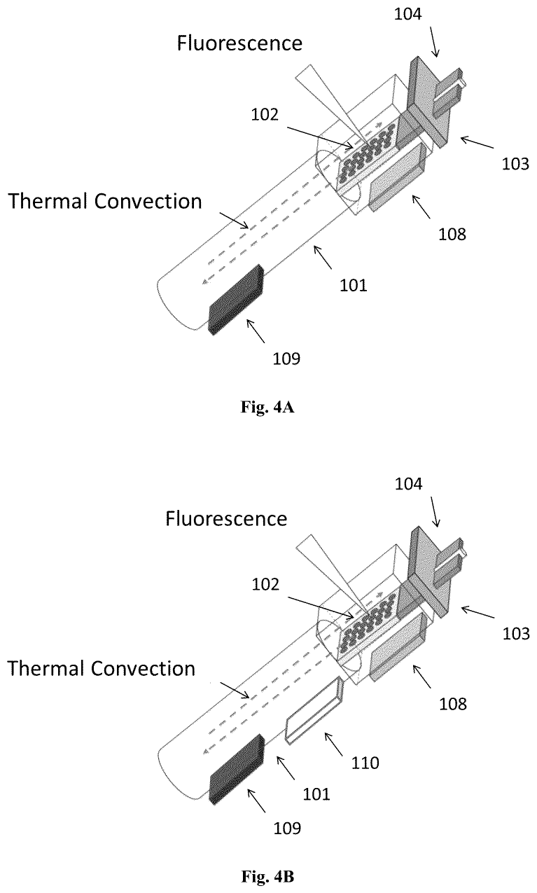

FIG. 4A is a schematic diagram of the overall structure of a detection device according to yet another embodiment of the present invention, and FIG. 4B is a schematic diagram of a partial structure of a detection device according to yet another embodiment of the present invention.

As shown in FIG. 4A, the detection device may further include a first heater 108 and a second heater 109 in addition to the various components shown in FIGS. 1A and 1B. The first heater 108 may be located at a first end of the reaction vessel 101. The second heater 109 may be located at a second end of the reaction vessel 101. The contact area of the reaction vessel 101 of the tubular structure with the first heater 108 and the second heater 109 is smaller than that of the annular convection chamber, and the heater matched with the tubular structure occupies a smaller space, and thus more reaction vessels 101 can be placed in the same given volume, so that high-throughput sample detection can be realized.

In one embodiment, both the first heater 108 and the second heater 109 are capable of heating the reaction vessel 101 such that a reaction mixture with a test sample injected inside can form convection flow between the first end and the second end of the reaction vessel 101, and such that one or more target molecules to be detected in the test sample can hybridize not only to the complementary primers in the reaction mixture for amplification, but also to the complementary nucleic acid probe molecules immobilized on the reaction surface of the reaction chip 102.

In one embodiment, the heating of both the first heater 108 and the second heater 109 may be controlled separately such that the temperature at the first end of the reaction vessel 101 may be lower than the temperature at the second end of the reaction vessel 101. It is understood that the higher the heating temperature, the lower the density of the heated reaction mixture with the test sample (lower specific gravity), and the lower the heating temperature, the higher the density of the heated reaction mixture with the test sample (higher specific gravity). In this way, the reaction mixture with the test sample at the first end of the reaction vessel 101 and the reaction mixture with the test sample at the second end of the reaction vessel 101 can form thermal convection so that liquids are continuously flowed and mixed, facilitating the hybridization process to be described in the following.

In one embodiment, the heating of the first heater 108 may be controlled so that the free target molecules in the test sample may hybridize with the free primers as well as with the complementary nucleic acid probe molecules immobilized on the reaction surface of the reaction chip 102, and the heating of the second heater 109 may be controlled such that double stranded target molecules in the test sample may be denatured into single stranded molecules and flow to the first end due to the low specific gravity of the second end, achieving exponential amplification.

In one embodiment, the heating of the first heater 108 may be controlled such that the temperature at the first end of the reaction vessel 101 may be between 30° C. and 75° C., and the heating of the second heater 109 may be controlled such that the temperature at the second end of the reaction vessel 101 may be between 35° C. and 110° C. (not boiling nor bubbling at <110° C. and 1.5 atmospheres). It is noted that in the existing process the temperature of the high heat zone is less than 100° C., such as 95° C., whereas the high heat zone temperature of the present invention may be greater than or equal to 100° C., such as 110° C., since the reaction vessel employed in the present invention is capable of withstanding high pressures, i.e. the liquid will not boil at temperatures greater than 100° C., and the enzyme employed in the present invention is capable of withstanding high temperatures, i.e. the enzyme will not denature at temperatures greater than 100° C.

In another embodiment, the heating of the first heater 108 may be controlled such that the temperature at the first end of the reaction vessel 101 may be between 35° C. and 75° C., and the heating of the second heater 109 may be controlled such that the temperature at the second end of the reaction vessel 101 may be between 75° C. and 110° C.

In yet another embodiment, the heating of the first heater 108 may be controlled such that the temperature at the first end of the reaction vessel 101 may be 65° C., and the heating of the second heater 109 may be controlled such that the temperature at the second end of the reaction vessel 101 may be 98° C.

In one embodiment, the first heater 108 and the second heater 109 may each be arranged outside the reaction space. In another embodiment, the first heater 108 and the second heater 109 may each be arranged inside the reaction space. In still another embodiment, one of the first heater 108 and the second heater 109 may be arranged outside the reaction space, and the other of the first heater 108 and the second heater 109 may be arranged inside the reaction space. As shown in FIG. 4A, the first heater 108 and the second heater 109 may each be arranged outside the reaction space.

However, the first heater 108 may be integrated into the reaction chip 102 to be arranged inside the reaction space, while the second heater 109 is still arranged outside the reaction space.

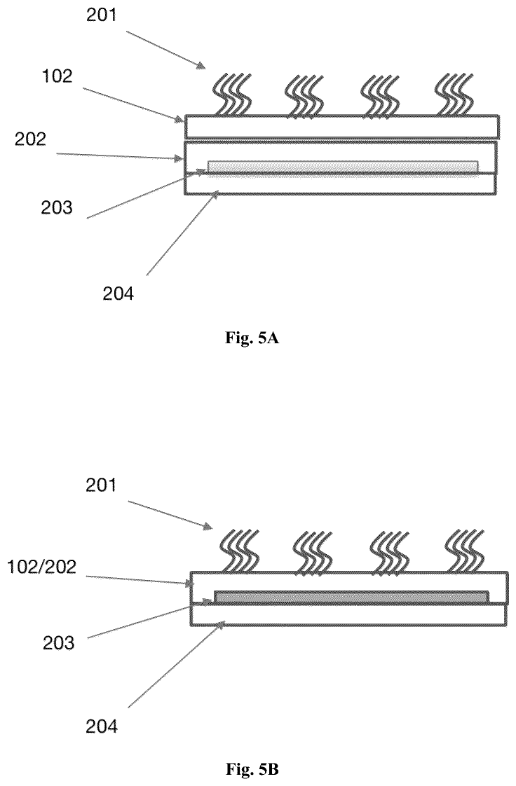

FIG. 5A to 5B are schematic diagrams illustrating an integration of the first heater and the reaction chip in the detection device according to an embodiment of the present invention, respectively.

As shown in FIG. 5A, the first heater 108 may include an insulating layer 202 and a base layer 204 with a heating layer 203 there between, and the reaction chip 102 having the nucleic acid probes 201 immobilized on the reaction surface is separately provided to the insulating layer 202.

As shown in FIG. 5B, the first heater 108 may include an insulating layer 202 and a base layer 204 with a heating layer 203 therebetween, and the reaction chip 102 having the nucleic acid probes 201 fixed on the reaction surface is integrally formed with the insulating layer 202.

In one embodiment, the first heater 108 and the second heater 109 may be a single heater to be arranged on the same side or different sides of the reaction vessel 101, respectively. In another embodiment, the first heater 108 and the second heater 109 may be dual heaters arranged on both sides of the reaction vessel 101, respectively. In still another embodiment, the first heater 108 and the second heater 109 may be ring heaters to be arranged around the reaction vessel 101, respectively.

In one embodiment, each of the first heater 108 and the second heater 109 may be coupled to the reaction vessel 101 in a manner that includes contact heat conduction, radiation, thermal convection, electromagnetic induction, and the like.

In one embodiment, the first heater 108 and the second heater 109 may take the form of resistive heaters, PET (Polyethylene terephthalate) heaters, PI (polyimide) heaters, or silicone heaters, among others. As an example, the first heater 108 may take the form of a resistive heater and may be integrated into the reaction chip 102 to be arranged inside the reaction space, and the second heater 109 may take the form of a PET heater, a PI heater, or a silicone heater and may be arranged outside the reaction space.

In one embodiment, the first heater 108 and the second heater 109 may be provided corresponding to one reaction vessel 101. In another embodiment, the first heater 108 and the second heater 109 may be provided corresponding to a plurality of reaction vessels 101, so that several to several tens of reactions may be simultaneously controlled, and high throughput sample detection may be achieved.

Returning to FIG. 4B, as shown in FIG. 4B, the detection device may further include a third heater 110 in addition to the various components shown in FIG. 4A, wherein the third heater 110 may be one or more heaters, and the third heater 110 may be located between the first end and the second end of the reaction vessel 101. The contact area of the reaction vessel 101 of the tubular structure with the first heater 108, the second heater 109 and the third heater 110 is smaller than that of the annular convection chamber, and the heater matched with the tubular structure occupies a smaller space, and thus more reaction vessels 101 can be placed for the same given volume, so that high-throughput sample detection can be achieved.

In one embodiment, the first heater 108 and the second heater 109 plus the third heater 110 are capable of heating the reaction vessel 101 such that a reaction mixture with a test sample injected inside can form convection flow between the first end and the second end of the reaction vessel 101 under the action of thermal convection, and such that one or more target molecules to be detected in the test sample can hybridize not only to the complementary primers in the reaction mixture to enable amplification, but also to the complementary nucleic acid probe molecules immobilized on the reaction surface of the reaction chip 102.

In one embodiment, the heating of the first heater 108, the second heater 109, and the third heater 110 may be controlled separately such that the temperature at the first end of the reaction vessel 101 may be lower than the temperature at the second end of the reaction vessel 101. It is understood that the higher the heating temperature, the lower the density of the heated reaction mixture with the test sample (lower specific gravity), and the lower the heating temperature, the higher the density of the heated reaction mixture with the test sample (higher specific gravity). In this way, the reaction mixture with the test sample at the first end of the reaction vessel 101 and the reaction mixture with the test sample at the second end of the reaction vessel 101 can form thermal convection so that the liquid can continuously flow and mix, facilitating the hybridization process to be described later.

In one embodiment, the heating of the first heater 108, the second heater 109 and the third heater 110 may be controlled separately so that free target molecules in the sample can hybridize with the free primers and complementary nucleic acid probe molecules immobilized on the reaction surface of the reaction chip 102, and the double-stranded target molecules in the sample can be denatured into single-stranded molecules, and flow to the first end due to the low specific gravity of the second end, thereby achieving exponential amplification.

In one embodiment, the heating of the first heater 108, the second heater 109, and the third heater 110 may be controlled separately such that the temperature at the first end of the reaction vessel 101 may be between 30° C. and 75° C. and such that the temperature at the second end of the reaction vessel 101 may be between 35° C. and 110° C. (not boiling nor bubbling at <110° C. and 1.5 atmospheres). It is noted that in the existing process the temperature of the high heat zone is less than 100° C., such as 95° C., whereas the high heat zone temperature of the present invention may be greater than or equal to 100° C., such as 110° C., since the reaction vessel employed in the present invention is capable of withstanding high pressures, i.e. the liquid will not boil at temperatures greater than 100° C., and the enzyme employed in the present invention is capable of withstanding high temperatures, i.e. the enzyme will not denature at temperatures greater than 100° C.

In another embodiment, the heating of the first heater 108, the second heater 109, and the third heater 110 may be controlled separately such that the temperature at the first end of the reaction vessel 101 may be 35° C. to 75° C. and such that the temperature at the second end of the reaction vessel 101 may be 75° C. to 110° C.

In yet another embodiment, the heating of the first heater 108, the second heater 109, and the third heater 110 may be controlled separately such that the temperature at the first end of the reaction vessel 101 may be 65° C. and such that the temperature at the second end of the reaction vessel 101 may be 98° C.

In one embodiment, the first heater 108, the second heater 109, and the third heater 110 may all be arranged outside the reaction space. In another embodiment, the first heater 108, the second heater 109, and the third heater 110 may all be arranged inside the reaction space. In still another embodiment, a part of the first heater 108, the second heater 109, and the third heaters 110 may be arranged outside the reaction space, and another part of the first heater 108, the second heater 109 and the third heater 110 may be arranged inside the reaction space. As shown in FIG. 4B, the first heater 108, the second heater 109, and the third heater 110 may be all arranged outside the reaction space.

However, the first heater 108 may be integrated into the reaction chip 102 to be arranged inside the reaction space, while the second heater 109 and the third heater 110 are still arranged outside the reaction space.

In one embodiment, the first heater 108, the second heater 109, and the third heater 110 may be a single heater to be arranged on the same side or different sides of the reaction vessel 101, respectively. In another embodiment, the first heater 108, the second heater 109, and the third heater 110 may be dual heaters to be arranged on both sides of the reaction vessel 101, respectively. In still another embodiment, the first heater 108, the second heater 109, and the third heater 110 may be ring heaters to be arranged around the reaction vessel 101, respectively.

In one embodiment, each of the first heater 108, the second heater 109, and the third heater 110 may be coupled to the reaction vessel 101 in a manner that includes contact heat conduction, radiation, thermal convection, electromagnetic induction, and the like.

In one embodiment, the first heater 108, the second heater 109, and the third heater 110 may take the form of resistive heaters, PET heaters, PI heaters, or silicone heaters, among others. As an example, the first heater 108 may take the form of a resistive heater and may be integrated into the reaction chip 102 to be arranged inside the reaction space, and the second heater 109 and the third heater 110 may take the form of a PET heater, a PI heater, or a silica gel heater and may be arranged outside the reaction space.

In one embodiment, the first heater 108, the second heater 109, and the third heater 110 may be provided corresponding to one reaction vessel 101. In another embodiment, the first heater 108, the second heater 109, and the third heater 110 may be provided corresponding to a plurality of reaction vessels 101, so that several to several tens of reactions may be simultaneously controlled, achieving high throughput sample detection.

In one embodiment, the plural types of target molecules may include one or more of RNA molecules or DNA molecules, RNA fragments in an RNA genome or DNA fragments in a DNA genome, and variant structures in an RNA molecule or a DNA molecule.

In one embodiment, the variant structure may include a single base polymorphism (Single Nucleotide Polymorphisms, SNP).

In one embodiment, the plural types of target molecules may include one or more of viral RNA nucleic acid molecules and viral DNA nucleic acid molecules. In other words, the plural types of target molecules may include only viral RNA nucleic acid molecules, only viral DNA nucleic acid molecules, or both viral RNA nucleic acid molecules and viral DNA nucleic acid molecules.

The RNA virus may include one or more of 14 common respiratory RNA viruses such as InfA, H1N1 influenza A2009, H3N2 influenza A, HPIV1, HPIV2, HPIV3, HPIV4, hMPV, AdV, RSV, BOV, SARS coronavirus SARS-COV, MERS-COV and SARS-COV-2.

The DNA virus may include one or more of 8 common human herpesviruses, such as human herpesvirus HSV-1, human herpesvirus HSV-2, human herpesvirus VZV, human herpesvirus CMV, human herpesvirus EBV, human herpesvirus HHV-6, human herpesvirus HHV-7, and human herpesvirus HHV-8.

In one embodiment, the plural types of target molecules may be of human, animal, plant, microbial or synthetic origin, wherein the microorganisms may include one or more of viruses, bacteria and fungi.

### Example 2

A second embodiment of the present invention provides a light guide detection method for a detection device, which has been described in the above first embodiment, and may comprise:

injecting the reaction mixture with the test sample into the interior of the reaction space provided by the reaction vessel, wherein said test sample may include one or more target molecules to be detected;
emitting excitation light by the light source on to the reaction chip via the light guide assembly to generate an evanescent wave on the reaction surface of the reaction chip such that a fluorescent signal can be generated after hybridization of nucleic acid molecules formed by amplification of one or more target molecules within the reaction vessel with complementary nucleic acid probe molecules immobilized on the reaction surface of the reaction chip;
detecting the fluorescent signal by a fluorescent signal detector;
determining the types of one or more target molecules that hybridize with the complementary nucleic acid probe molecules based on the types of said complementary nucleic acid probes where fluorescent signals are detected.

In one embodiment, the excitation light may be directed into the reaction chip by the coupler at an angle of total internal reflection. More specifically, the excitation light may be converted into a sheet-light by an optical converter through an optical fiber, and then irradiated into the reaction chip at a total internal reflection angle via a coupler.

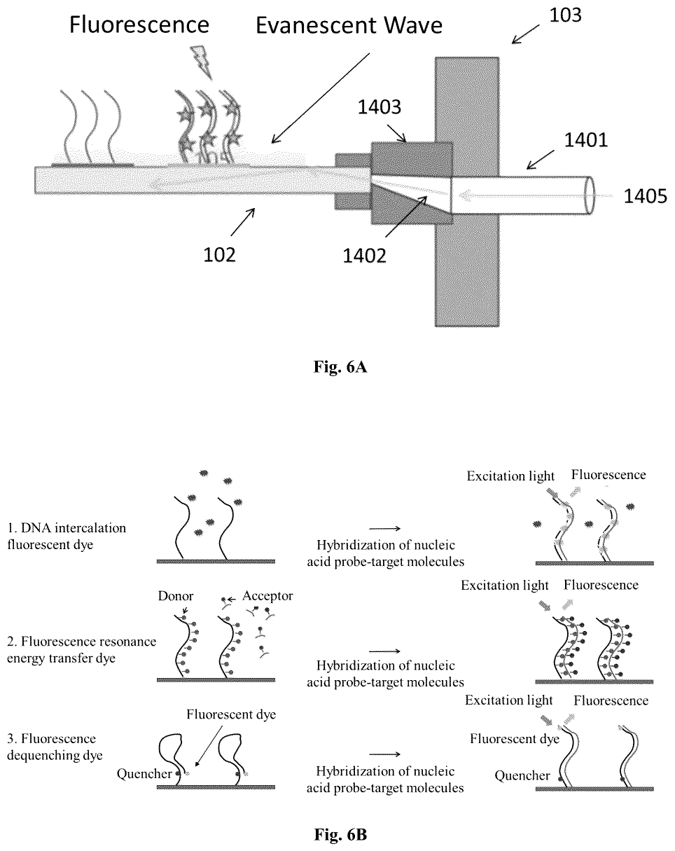

FIG. 6A to 6B are respectively a principle diagram and a schematic diagram of a light guide detection method for a detection device according to an embodiment of the present invention.

As shown in FIG. 6A, excitation light emitted by the light source 1405 may pass through the optical fiber 1401, be converted into a sheet-light by the light converter 1402, and be irradiated into the reaction chip 102 via the coupler 1403 at a total internal reflection angle, so that an evanescent wave may be generated on the reaction surface of the reaction chip 102, and a fluorescent signal may be generated after hybridization of a target molecule with a complementary nucleic acid probe molecule.

In one embodiment, referring to FIGS. 1A and 1B, excitation light may be coupled into an optical fiber 1401. More specifically, excitation light may be coupled into the optical fiber 1401 by a condenser lens 1404.

The Evanescent wave can also be called as Evanescent field, surface wave, and the like, and compared with the excitation light that irradiates directly on a reaction chip, the invention adopts a light guide assembly-Evanescent wave method that can effectively prevent the reaction mixture from being affected by light source irradiation, reduces phototoxic effect, and effectively avoid interference of background fluorescence, hereby improving the reliability of reading results.

In one embodiment, referring to FIG. 6A, the evanescent wave propagates parallelly to the reaction surface of the reaction chip 102, ranging from the reaction surface of the reaction chip 102 to the first thickness, wherein the intensity of the evanescent wave decays exponentially with increasing thickness. In another embodiment, the first thickness may be 100 nm.

In one embodiment, the light guide detection method may further include heating the reaction vessel by the first heater and the second heater, so that the reaction mixture with the test sample can form convection flow between the first end and the second end of the reaction vessel under the action of thermal convection, and so that one or more target molecules to be detected in the reaction mixture can hybridize with complementary primers in the reaction mixture to achieve amplification, and can hybridize with complementary nucleic acid probe molecules immobilized on the reaction surface of the reaction chip.

In one embodiment, the heating of both the first heater and the second heater may be controlled separately such that the temperature at the first end of the reaction vessel may be lower than the temperature at the second end of the reaction vessel. It is understood that the higher the heating temperature, the lower the density of the heated reaction mixture with the test sample (lower specific gravity), and the lower the heating temperature, the higher the density of the heated reaction mixture with the test sample (higher specific gravity). In this way, the reaction mixture with the test sample at the first end of the reaction vessel and the reaction mixture with the test sample at the second end of the reaction vessel can form thermal convection so that liquids are continuously flowed and mixed, facilitating the hybridization process to be described later.

In one embodiment, the heating of the first heater may be controlled so that the free target molecules in the reaction mixture can hybridize with the free primers and complementary nucleic acid probe molecules immobilized on the reaction surface of the reaction chip, and the heating of the second heater may be controlled such that double stranded target molecules in the reaction mixture may be denatured into single stranded molecules and flow to the first end due to the low specific gravity of the second end, whereby achieving exponential amplification.

In one embodiment, the heating of the first heater may be controlled such that the temperature at the first end of the reaction vessel may be between 30° C. and 75° C., and the heating of the second heater may be controlled such that the temperature at the second end of the reaction vessel may be between 35° C. and 110° C. (not boiling nor bubbling at <110° C. and 1.5 atmospheres). It is noted that in the existing process the temperature of the high heat zone is less than 100° C., such as 95° C., whereas the high heat zone temperature of the present invention may be greater than or equal to 100° C., such as 110° C., since the reaction vessel employed in the present invention is capable of withstanding high pressures, i.e. the liquid will not boil at temperatures greater than 100° C., and the enzyme employed in the present invention is capable of withstanding high temperatures, i.e. will not denature at temperatures greater than 100° C.

In another embodiment, the heating of the first heater may be controlled such that the temperature at the first end of the reaction vessel may be between 35° C. and 75° C., and the heating of the second heater may be controlled such that the temperature at the second end of the reaction vessel may be between 75° C. and 110° C.

In yet another embodiment, the heating of the first heater may be controlled such that the temperature at the first end of the reaction vessel may be 65° C., and the heating of the second heater may be controlled such that the temperature at the second end of the reaction vessel may be 98° C.

In one embodiment, the light guide detection method may further include heating the reaction vessel by one or more third heaters.

In one embodiment, the heating of the first heater, the second heater and the third heater may be controlled separately such that the temperature at the first end of the reaction vessel may be lower than the temperature at the second end of the reaction vessel. It is understood that the higher the heating temperature, the lower the density of the heated reaction mixture with the test sample (lower specific gravity), and the lower the heating temperature, the higher the density of the heated reaction mixture with the test sample (higher specific gravity). In this way, the reaction mixture with the test sample at the first end of the reaction vessel and the reaction mixture with the test sample at the second end of the reaction vessel can form thermal convection so that liquids can continuously flow and mix, facilitating the hybridization process to be described later.

In one embodiment, the heating of the first heater, the second heater and the third heater may be controlled separately so that the free target molecules in the reaction mixture can hybridize with the free primers and complementary nucleic acid probe molecules immobilized on the reaction surface of the reaction chip, and double-stranded target molecules in the reaction mixture are denatured into single-stranded molecules and flow to the first end due to the low specific gravity of the second end, whereby achieving exponential amplification.

In one embodiment, the heating of the first, second and third heaters may be controlled separately such that the temperature at the first end of the reaction vessel may be between 30° C. and 75° C. and such that the temperature at the second end of the reaction vessel may be between 35° C. and 110° C. (not boiling nor bubbling at <110° C. and 1.5 atmospheres). It is noted that in the existing process the temperature of the high heat zone is less than 100° C., such as 95° C., whereas the high heat zone temperature of the present invention may be greater than or equal to 100° C., such as 110° C., since the reaction vessel employed in the present invention is capable of withstanding high pressures, i.e. the liquid will not boil at temperatures greater than 100° C., and the enzyme employed in the present invention is capable of withstanding high temperatures, i.e. will not denature at temperatures greater than 100° C.

In another embodiment, the heating of the first, second and third heaters may be controlled separately such that the temperature at the first end of the reaction vessel may be between 35° C. and 75° C. and such that the temperature at the second end of the reaction vessel may be between 75° C. and 110° C.

In yet another embodiment, the heating of the first, second and third heaters may be controlled separately such that the temperature at the first end of the reaction vessel may be 65° C. and such that the temperature at the second end of the reaction vessel may be 98° C.

In one embodiment, the reaction vessel may be vertically arranged, and in another embodiment, the reaction vessel may be obliquely arranged. As shown in FIGS. 1A and 1B, the reaction vessel 101 may be vertically arranged, and as shown in FIGS. 4A and 4B, the reaction vessel 101 may be obliquely arranged. In yet another embodiment, the reaction vessel is angled between 0° and 45° from vertical.

Herein, the reaction system refers to a liquid to be injected into the inside of a reaction space provided by a reaction vessel and for mixing with a test sample, resulting in a reaction mixture; in other words, a reaction system refers to all liquids inside the reaction space except for the test sample in the detection by the detection device. The reaction system or the reaction mixture, which may be used interchangeably, may have at least two functions, one is to provide a liquid enzymatic reaction environment for multiplex amplification and the other is to provide conditions for molecular hybridization of the target molecules with the nucleic acid probes.

In one embodiment, the reaction system may include a plural pairs of primers complementary to respective types of target molecules, and these primers may be used to hybridize with the complementary target molecules in the test sample to achieve amplification, thereby increasing the concentration of the target molecules via molecular amplification under the action of thermal convection, increasing the chance of hybridization between the molecules of nucleic acid probes immobilized on the reaction surface of the reaction chip and the target molecules. Molecular amplification may include Polymerase Chain Reaction (PCR), loop-mediated isothermal amplification (LAMP), nicking endonuclease isothermal amplification technique (NEAR), nucleic acid sequence dependent amplification (NASBA), rolling circle nucleic acid amplification (RCA), melting enzyme amplification (HDA), recombinase Polymerase Amplification (RPA), and enzymatic recombination isothermal amplification technique (ERA).

As shown in Table 1 below, For different types of target molecules, complementary primers may be prepared and added to the reaction system, and complementary nucleic acid probes may be prepared for immobilization onto the reaction surface of the reaction chip.

TABLE 1
Names of target molecules and corresponding primer sequences and nucleic acid
probe sequences

| Name of | | |
|---|---|---|
| target | | |
| molecules | Primer Sequences (5′→3′) | Probe Sequences (5′→3′) |
| InfA | F: AGACCAATYYTGTCACCTCTG | TACGCTCACCGTGCCC |
| | R: CGGGTCCCCATTCCCATT | AGTGAACG |
| H1N1 | F: GGTTTGAGATATTCCCCAARAC | TCATGATTCGAACAAA |
| 2009 | R: AGGATTTGCTGAGCTTTGGGTATG | GGTGTAAC |
| H3N2 | F: CGAAGCAAAGCCTACAGCAAC | GGATTATGCCTCCCTTA |
| | R: TGTCAAATTGTTCATTGTTTGGCAT | GGTCACTA |
| HPIV1 | F: ACTAGGTGTGACAGACACAGCAA | TCAGGAGGGGATGGTG |
| | R: TGTTGTCCCGATCAGCAGTGTC | CCTACCAT |
| HPIV2 | F: AATGGGCCACAATCAATCCT | GAAGCGGGATCTATCA |
| | R: ATAACATAGAGCCTGCCTTCTGC | CCTAGGCT |
| HPIV3 | F: TTCATCTGTATCCTCAGAGATCCC | GTTCGCACCAGGCAAC |
| | R: CTCATCTGAGCTTCAGCATCAC | TATCCTGCCA |
| HPIV4 | F: CTGAAGAGAGAMGRCTGGCYAAG | AGCAGCAGGGCAGGAT |
| | R: AGCTGGAGCAAATTCCATCAATTC | GTTAGAGA |
| hMPV | F: CATAYAARCATGCTATATTAAAAGAGTC | GATGTAGGCACCACAA |
| | R: GTTTCTTAGAATCTGCTGTACTCTCT | CTGCAGTGA |
| AdV | F: AGGACGCYTCGGAGTACCT | CTGGTGCAGTTCGCCC |
| | R: GCTAGGACCTCTATCAAGCACC | GTGCCACCG |
| RSV | F: TTAGCAAAGTCAAGYTGAATGATAC | TAAGGATCAGCTGCTG |
| | R: CTTTAAGTATCTTTATAGTGTCTTC | TCATCCAGC |
| BoV | F: GAAGAGACACTGGCAGACAACTC | TCCGACACAGTGGGGA |
| | R: AACATTAGCTAAGTGTCTACGGTAC | GAGAGGCTC |
| SARS- | F: GCCTCTCTTGTTCTTGCTCGC | CACTTGCTGCAACTTAT |
| CoV | R: CAGGTAAGCGTAAAACTCATC | CACACCGT |
| MERS- | F: GGCACTGAGGACCCACGTT | CTGAGCTTGCTCCTACA |
| CoV | R: TTGCGACATACCCATAAAAGCA | GCCAGTG |
| SARS- | F: CAACAATGGGGTTTTACAGGTAACCT | GTCCATGGTAATGCAC |
| CoV-2 | R: GTCAACACGCTTAACAAAGCACTC | ATGTAGCT |
| HSV-1 | F: CCTYCTCAGCAACACGCTC | ACACCTCCGAGAGCAG |
| | R: GAGGAGGTGGTCTTGATGC | AGCCGCA |
| HSV-2 | F: GGTCACCAACATGGTTCTG | ACTCTCCGCTCCACAAC |
| | R: ATACACAAGCCCAGCTCCC | GAGGAC |
| VZV | F: CGAGCTTAGTCGTCCACC | ACCGTCGCTTGGCTCAC |
| | R: GATTTGACCCTGTCTCATTAC | TTAACAG |
| CMV | F: CTACACCTCGCTGCTRGAC | TCCCACCCAGACTAGC |
| | R: GACATCTGAAACATRGCCGC | CCCCAGG |
| EBV | F: ACTGAGGAGGGCATGAAGC | TCCYTGCCCYAACCAC |
| | R: GAAGAGGAGGTGGTAAGCG | TACAGCAAAG |
| HHV-6 | F: AGTAAGACGGGATATAATGCC | TGCCTCCGTATCTKTAC |
| | R: TACACATCTGTGACGCTACC | GAATGC |
| HHV-7 | F: TCGGAACTCCTAATACGATTC | ACGCCCTATCGCAACA |
| | R: TGCCATAAGAAACAGGTACAG | CATTTCAC |
| HHV-8 | F: ACGCCCTATCGCAACACATTTCAC | TCGCTCCTGGATMACG |
| | R: GGGAAGTGTTCCTSCTGAG | GGGTCTC |

In one embodiment, the reaction system may include a primer and a DNA polymerase. In another embodiment, the reaction system may include 3 mM MgCl₂, 0.2 mM dNTP, a plurality of primers at a concentration ranged from 0.1 μM to 0.6 μM, a DNA polymerase at a concentration of 0.05 U/μl, a RNA reverse transcriptase at a concentration of 0.5 U/μl, DTT at a concentration of 1 mM, Tween-20 at a concentration of 0.05%, Tris-HCl at a pH of 8.8 and at a concentration of 25 mM, and K₂SO₄ at a concentration of 30 mM. In yet another embodiment, the DNA polymerase has a 3′→5′ exonuclease activity. By using a polymerase having a 3′→5′ exonuclease activity, the detection rates of virus variant strains can be increased, mis-paired bases of primers can be repaired, and high fidelity can be achieved, reducing nonspecifically amplified products, making the detection results more reliable.

In one embodiment, the volume of the reaction mixture may be 50 μl.

It will be appreciated that the various types of target molecules listed above are for illustration only and are not intended to be limiting. In fact, one skilled in the art can set other types of target molecules according to actual needs, and prepare primer sequences and nucleic acid probe sequences complementary to the target molecules, and adjust a reaction mixture to achieve multiplex amplification and multiplex detection of a plural types of target molecules simultaneously in the same liquid-phase reaction mixture.

In one embodiment, the fluorescent signal may be generated by direct excitation of fluorescent dyes, dye intercalation, fluorescence resonance energy transfer, or fluorescence dequenching.

As shown in FIG. 6B, for the dye intercalation method, a double-stranded DNA molecule-specific intercalating fluorescent dye, e.g., including EB, SYBR series, gold View series, gel Red, etc., is employed that does not emit fluorescence or emits weak fluorescence when the dye is free, but emits strong fluorescence, with an enhancement of 20 to 30 times, when the dye is interacted in double-helical DNA molecules, thereby specifically detecting the double-stranded DNA. These fluorochromes are added to the amplification reaction mixture before the detection reaction at a final concentration of 0.05-5.0 μM.

As shown in FIG. 6B, for the fluorescence resonance energy transfer method, fluorescent dyes that stimulate fluorescence resonance energy transfer through molecular hybridization are used. The dyes are classified into donor dyes and acceptor dyes. A nucleic acid probe molecule can be connected to a donor dye or an acceptor dye and a target molecule can be connected to an acceptor dye or a donor dye. The donor dye and the acceptor dye are brought close to each other only when a probe molecule hybridizes with the target molecule, thereby causing the acceptor dye to emit light when the donor dye is excited.

As shown in FIG. 6B, for the fluorescence dequenching method, a fluorescent dye is quenched by an adjacent quencher and does not emit light when a probe molecule is not hybridized with a target molecule. When a probe molecule hybridizes with the target molecule, the quencher molecule is removed or displaced so that the fluorescent dye is de-quenched and emits light, for example, in the case of TaqMan probe method, molecular beacon method, scorpion probe method, etc.

In one embodiment, the type of a nucleic acid probe may be identified by a predetermined location of the nucleic acid probe on the reaction surface of the reaction chip. As already described in the first embodiment above, when a molecule of a known nucleic acid probe at a given position on the reaction surface hybridizes with a complementary target molecule to form a double-stranded molecule, the presence of the complementary target molecule in the test sample can be identified. In the case where a plurality of nucleic acid probes complementary to a plural types of target molecules are immobilized on the reaction surface, it is possible to detect whether a plural types of target molecules are present in the test sample, thereby achieving a multiplex detection.

In one embodiment, the amount of target molecules can be calculated from the kinetic data of the signals. The signal kinetic data mainly uses the changes of fluorescence intensity with reaction time as the raw data; the raw data is fitted by a signal processing system according to a Logistic growth curve and other models; then a PCR reaction is determined to be positive or negative according to the fitting coefficient (R value or R2 Value) and the difference in fluorescence intensity between before and after the reaction.

The first embodiment is a device embodiment corresponding to the present embodiment, and the present embodiment can be implemented in cooperation with the first embodiment. The relevant technical details mentioned in the first embodiment are still valid in this embodiment, and will not be restated here to reduce repetition. Accordingly, the relevant technical details mentioned in the present embodiment can also be applied to the first embodiment.

While the invention has been shown and described with reference to certain preferred embodiments thereof, it will be understood by those skilled in the art that various changes in form and details may be made therein without departing from the spirit and scope of the invention.

1. A detection device, comprising:
a reaction vessel providing a reaction space and having a first end and a second end opposite said first end, wherein the first end of said reaction vessel is an open end and said second end of said reaction vessel is a closed end, wherein said reaction vessel is used for nucleic acid amplification reactions;
a reaction chip arranged inside the reaction space and at said first end of said reaction vessel, wherein a plural types of nucleic acid probes complementary to a plural types of target molecules, respectively, are immobilized on a reaction surface of said reaction chip, wherein said nucleic acid probes are used to detect nucleic acid molecules formed by amplifying said target molecules in said reaction vessel;
a cover detachably provided on the first end of said reaction vessel to close said reaction space;
a light guide assembly passing through said cover from the outside of the reaction space into the inside of said reaction space, wherein said light guide assembly is connected to the reaction chip.

2. The device of claim 1, wherein the light guide assembly comprises an optical fiber, an optical converter, and a coupler, wherein the optical fiber is connected to the optical converter, and the optical converter connects the excitation light to the reaction chip via the coupler.

3. The device of claim 2, wherein the light guide assembly further comprises a condenser lens and a light source, wherein the condenser lens can direct excitation light emitted by the light source into the optical fiber.

4. The device of claim 1, wherein the reaction chip is fixed to the cover vertically or horizontally via the light guide assembly.

5. (canceled)

6. The device of claim 1, further comprising a fluorescent signal detector that detects a fluorescent signal on the reaction chip.

7. The device of claim 6, further comprising a computing-control module that controls the light guide assembly and the fluorescence signal detector and outputs a detection result of the detection device based on the detection of the fluorescence signal detector.

8. The device of claim 1, wherein the device further comprises:
a first heater located at the first end of the reaction vessel;
a second heater located at the second end of the reaction vessel.

9. The device of claim 8, wherein both the first heater and the second heater are heated, such that the temperature at the first end of the reaction vessel is 30° C. to 75° C. and such that the temperature at the second end of the reaction vessel is 35° C. to 110° C.

10. The device of claim 8, wherein the first heater and the second heater are each a single heater, a dual heater, or a ring heater.

11. The device of claim 8, wherein the reaction vessel is a tubular structure, the first end and the second end of the reaction vessel are arranged opposite each other in a length direction of the tubular structure, the first end and the second end of the reaction vessel are arranged concentrically or non-concentrically, and cross sections of the first end and the second end of the reaction vessel are the same or different.

12. The device of claim 11, wherein the cross sections of the first end and the second end of the reaction vessel consist of at least one of curvilinear and rectilinear sides, respectively.

13. (canceled)

14. The device of claim 11, wherein the reaction surface of the reaction chip is directed in a radial direction of the tubular structure or in the length direction of the tubular structure and toward the second end of the reaction vessel.

15. (canceled)

16. The device of claim 8, wherein the first heater is integrated into the reaction chip.

17. The device of claim 1, wherein the plural types of target molecules comprise one or more of RNA molecules or DNA molecules, RNA fragments in an RNA genome or DNA fragments in a DNA genome, and variant structures in an RNA molecule or a DNA molecule.

18. (canceled)

19. A light guide detection method for use in a detection device according to claim 1, the method comprising:
injecting a reaction mixture with a test sample into the interior of a reaction space provided by a reaction vessel, said test sample comprising one or more target molecules to be detected;
emitting excitation light by a light source and guiding the excitation light to a reaction chip via a light guide assembly to generate an evanescent wave on a reaction surface of the reaction chip, such that nucleic acid molecules formed by amplifying one or more of the target molecules within the reaction vessel can generate a fluorescent signal after hybridization of the nucleic acid molecules with complementary nucleic acid probe molecules immobilized on the reaction surface of the reaction chip;
detecting the fluorescent signals by a fluorescent signal detector;
determining the types of one or more target molecules that hybridize with the complementary nucleic acid probe molecules based on locations or types of said complementary nucleic acid probes where fluorescent signals are detected.

20. The method of claim 19, wherein the excitation light is directed into the reaction chip by a coupler at a total internal reflection angle.

21. The method of claim 19, further comprising heating the reaction vessel by a first heater and a second heater such that the reaction mixture with a test sample forms convection flow between a first end and a second end of the reaction vessel, and such that the one or more target molecules to be detected in the test sample can hybridize not only to the complementary primers in the reaction system to enable amplification, but also to the complementary nucleic acid probe molecules immobilized on the reaction surface of the reaction chip.

22. The method of claim 21, wherein heating of both the first and second heaters are controlled separately such that the temperature at the first end of the reaction vessel is 30° C. to 75° C. and such that the temperature at the second end of the reaction vessel is 35° C. to 110° C.

23. The method of claim 21, wherein the reaction system comprises a primer and a DNA polymerase.

24. The method of claim 23, wherein the DNA polymerase has 3′→5′ exonuclease activity.

25. (canceled)


SEQUENCE LISTING (66 sequences)

SEQ ID NO: 1 - DNA, 21 bp, synthetic construct
  source 1..21: mol_type = other DNA
agaccaatyytgtcacctctg

SEQ ID NO: 2 - DNA, 18 bp, synthetic construct
  source 1..18: mol_type = other DNA
cgggtccccattcccatt

SEQ ID NO: 3 - DNA, 22 bp, synthetic construct
  source 1..22: mol_type = other DNA
ggtttgagatattccccaarac

SEQ ID NO: 4 - DNA, 24 bp, synthetic construct
  source 1..24: mol_type = other DNA
aggatttgctgagctttgggtatg

SEQ ID NO: 5 - DNA, 21 bp, synthetic construct
  source 1..21: mol_type = other DNA
cgaagcaaagcctacagcaac

SEQ ID NO: 6 - DNA, 25 bp, synthetic construct
  source 1..25: mol_type = other DNA
tgtcaaattgttcattgtttggcat

SEQ ID NO: 7 - DNA, 23 bp, synthetic construct
  source 1..23: mol_type = other DNA
actaggtgtgacagacacagcaa

SEQ ID NO: 8 - DNA, 22 bp, synthetic construct
  source 1..22: mol_type = other DNA
tgttgtcccgatcagcagtgtc

SEQ ID NO: 9 - DNA, 20 bp, synthetic construct
  source 1..20: mol_type = other DNA
aatgggccacaatcaatcct

SEQ ID NO: 10 - DNA, 23 bp, synthetic construct
  source 1..23: mol_type = other DNA
ataacatagagcctgccttctgc

SEQ ID NO: 11 - DNA, 24 bp, synthetic construct
  source 1..24: mol_type = other DNA
ttcatctgtatcctcagagatccc

SEQ ID NO: 12 - DNA, 22 bp, synthetic construct
  source 1..22: mol_type = other DNA
ctcatctgagcttcagcatcac

SEQ ID NO: 13 - DNA, 23 bp, synthetic construct
  source 1..23: mol_type = other DNA
ctgaagagagamgrctggcyaag

SEQ ID NO: 14 - DNA, 24 bp, synthetic construct
  source 1..24: mol_type = other DNA
agctggagcaaattccatcaattc

SEQ ID NO: 15 - DNA, 28 bp, synthetic construct
  source 1..28: mol_type = other DNA
catayaarcatgctatattaaaagagtc

SEQ ID NO: 16 - DNA, 26 bp, synthetic construct
  source 1..26: mol_type = other DNA
gtttcttagaatctgctgtactctct

SEQ ID NO: 17 - DNA, 19 bp, synthetic construct
  source 1..19: mol_type = other DNA
aggacgcytcggagtacct

SEQ ID NO: 18 - DNA, 22 bp, synthetic construct
  source 1..22: mol_type = other DNA
gctaggacctctatcaagcacc

SEQ ID NO: 19 - DNA, 25 bp, synthetic construct
  source 1..25: mol_type = other DNA
ttagcaaagtcaagytgaatgatac

SEQ ID NO: 20 - DNA, 25 bp, synthetic construct
  source 1..25: mol_type = other DNA
ctttaagtatctttatagtgtcttc

SEQ ID NO: 21 - DNA, 23 bp, synthetic construct
  source 1..23: mol_type = other DNA
gaagagacactggcagacaactc

SEQ ID NO: 22 - DNA, 25 bp, synthetic construct
  source 1..25: mol_type = other DNA
aacattagctaagtgtctacggtac

SEQ ID NO: 23 - DNA, 21 bp, synthetic construct
  source 1..21: mol_type = other DNA
gcctctcttgttcttgctcgc

SEQ ID NO: 24 - DNA, 21 bp, synthetic construct
  source 1..21: mol_type = other DNA
caggtaagcgtaaaactcatc

SEQ ID NO: 25 - DNA, 19 bp, synthetic construct
  source 1..19: mol_type = other DNA
ggcactgaggacccacgtt

SEQ ID NO: 26 - DNA, 22 bp, synthetic construct
  source 1..22: mol_type = other DNA
ttgcgacatacccataaaagca

SEQ ID NO: 27 - DNA, 26 bp, synthetic construct
  source 1..26: mol_type = other DNA
caacaatggggttttacaggtaacct

SEQ ID NO: 28 - DNA, 24 bp, synthetic construct
  source 1..24: mol_type = other DNA
gtcaacacgcttaacaaagcactc

SEQ ID NO: 29 - DNA, 19 bp, synthetic construct
  source 1..19: mol_type = other DNA
cctyctcagcaacacgctc

SEQ ID NO: 30 - DNA, 19 bp, synthetic construct
  source 1..19: mol_type = other DNA
gaggaggtggtcttgatgc

SEQ ID NO: 31 - DNA, 19 bp, synthetic construct
  source 1..19: mol_type = other DNA
ggtcaccaacatggttctg

SEQ ID NO: 32 - DNA, 19 bp, synthetic construct
  source 1..19: mol_type = other DNA
atacacaagcccagctccc

SEQ ID NO: 33 - DNA, 18 bp, synthetic construct
  source 1..18: mol_type = other DNA
cgagcttagtcgtccacc

SEQ ID NO: 34 - DNA, 21 bp, synthetic construct
  source 1..21: mol_type = other DNA
gatttgaccctgtctcattac

SEQ ID NO: 35 - DNA, 19 bp, synthetic construct
  source 1..19: mol_type = other DNA
ctacacctcgctgctrgac

SEQ ID NO: 36 - DNA, 20 bp, synthetic construct
  source 1..20: mol_type = other DNA
gacatctgaaacatrgccgc

SEQ ID NO: 37 - DNA, 19 bp, synthetic construct
  source 1..19: mol_type = other DNA
actgaggagggcatgaagc

SEQ ID NO: 38 - DNA, 19 bp, synthetic construct
  source 1..19: mol_type = other DNA
gaagaggaggtggtaagcg

SEQ ID NO: 39 - DNA, 21 bp, synthetic construct
  source 1..21: mol_type = other DNA
agtaagacgggatataatgcc

SEQ ID NO: 40 - DNA, 20 bp, synthetic construct
  source 1..20: mol_type = other DNA
tacacatctgtgacgctacc

SEQ ID NO: 41 - DNA, 21 bp, synthetic construct
  source 1..21: mol_type = other DNA
tcggaactcctaatacgattc

SEQ ID NO: 42 - DNA, 21 bp, synthetic construct
  source 1..21: mol_type = other DNA
tgccataagaaacaggtacag

SEQ ID NO: 43 - DNA, 24 bp, synthetic construct
  source 1..24: mol_type = other DNA
acgccctatcgcaacacatttcac

SEQ ID NO: 44 - DNA, 19 bp, synthetic construct
  source 1..19: mol_type = other DNA
gggaagtgttcctsctgag

SEQ ID NO: 45 - DNA, 24 bp, synthetic construct
  source 1..24: mol_type = other DNA
tacgctcaccgtgcccagtgaacg

SEQ ID NO: 46 - DNA, 24 bp, synthetic construct
  source 1..24: mol_type = other DNA
tcatgattcgaacaaaggtgtaac

SEQ ID NO: 47 - DNA, 25 bp, synthetic construct
  source 1..25: mol_type = other DNA
ggattatgcctcccttaggtcacta

SEQ ID NO: 48 - DNA, 24 bp, synthetic construct
  source 1..24: mol_type = other DNA
tcaggaggggatggtgcctaccat

SEQ ID NO: 49 - DNA, 24 bp, synthetic construct
  source 1..24: mol_type = other DNA
gaagcgggatctatcacctaggct

SEQ ID NO: 50 - DNA, 26 bp, synthetic construct
  source 1..26: mol_type = other DNA
gttcgcaccaggcaactatcctgcca

SEQ ID NO: 51 - DNA, 24 bp, synthetic construct
  source 1..24: mol_type = other DNA
agcagcagggcaggatgttagaga

SEQ ID NO: 52 - DNA, 25 bp, synthetic construct
  source 1..25: mol_type = other DNA
gatgtaggcaccacaactgcagtga

SEQ ID NO: 53 - DNA, 25 bp, synthetic construct
  source 1..25: mol_type = other DNA
ctggtgcagttcgcccgtgccaccg

SEQ ID NO: 54 - DNA, 25 bp, synthetic construct
  source 1..25: mol_type = other DNA
taaggatcagctgctgtcatccagc

SEQ ID NO: 55 - DNA, 25 bp, synthetic construct
  source 1..25: mol_type = other DNA
tccgacacagtggggagagaggctc

SEQ ID NO: 56 - DNA, 25 bp, synthetic construct
  source 1..25: mol_type = other DNA
cacttgctgcaacttatcacaccgt

SEQ ID NO: 57 - DNA, 24 bp, synthetic construct
  source 1..24: mol_type = other DNA
ctgagcttgctcctacagccagtg

SEQ ID NO: 58 - DNA, 24 bp, synthetic construct
  source 1..24: mol_type = other DNA
gtccatggtaatgcacatgtagct

SEQ ID NO: 59 - DNA, 23 bp, synthetic construct
  source 1..23: mol_type = other DNA
acacctccgagagcagagccgca

SEQ ID NO: 60 - DNA, 23 bp, synthetic construct
  source 1..23: mol_type = other DNA
actctccgctccacaacgaggac

SEQ ID NO: 61 - DNA, 24 bp, synthetic construct
  source 1..24: mol_type = other DNA
accgtcgcttggctcacttaacag

SEQ ID NO: 62 - DNA, 23 bp, synthetic construct
  source 1..23: mol_type = other DNA
tcccacccagactagcccccagg

SEQ ID NO: 63 - DNA, 26 bp, synthetic construct
  source 1..26: mol_type = other DNA
tccytgcccyaaccactacagcaaag

SEQ ID NO: 64 - DNA, 23 bp, synthetic construct
  source 1..23: mol_type = other DNA
tgcctccgtatctktacgaatgc

SEQ ID NO: 65 - DNA, 24 bp, synthetic construct
  source 1..24: mol_type = other DNA
acgccctatcgcaacacatttcac

SEQ ID NO: 66 - DNA, 23 bp, synthetic construct
  source 1..23: mol_type = other DNA
tcgctcctggatmacggggtctc